(19) United States
(12) Patent Application Publication
(10) Pub. No.: US 2007/0161042 A1
(43) Pub. Date: Jul. 12, 2007

(54) Methods for characterizing molecular interactions

(75) Inventors: Robert Zuk, Atherton, CA (US); Krista Witte, Hayward, CA (US); Bettina Heidecker, Mountain View, CA (US)

Correspondence Address: FENWICK & WEST LLP, SILICON VALLEY CENTER, 801 CALIFORNIA STREET, MOUNTAIN VIEW, CA, 94041, US

(73) Assignee: ForteBio, Inc., Menlo Park, CA (US)

(21) Appl. No.: 11/330,925

(22) Filed: Jan. 11, 2006

(51) Int. Cl. G01N 33/53

(52) U.S. Cl. 435/7.1

(57) ABSTRACT

Methods are provided for measuring rate constants for high affinity molecular interactions using an assay format for determining dissociation rates in liquid phase. The invention uses a biosensor that at selected time intervals is contacted with a sample solution to estimate the ratio of bound vs. free ligand. Dissociation rate constants determined according to the methods of the invention more closely mimic in vivo binding constants and avoid diffusional barrier artifacts that accompany measurements performed using solid phase devices. The methods of the invention provide further advantage by not requiring continuous measurements be made on a biosensor instrument thus leaving it available to process other samples. The methods permit accurate determination of dissociation rates of reactions for which dissociation slowly occurs over intervals of hours to days or more.

### CROSS REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The invention relates to methods and compositions useful for characterizing high affinity molecular reactions.

2. Description of the Related Art

The goal for many biotechnology companies producing therapeutic monoclonal antibodies is to develop antibodies with high affinity binding to the biological target. Most therapeutic antibodies have affinity constants in the nanomolar range and much research is devoted to improving the affinity to approach the picomolar range. Instruments capable of making kinetic or real-time measurements of binding reactions (such as, e.g., etalon fiber-based systems available from FortéBio, Inc., and surface plasmon-resonance based instruments available from Biacore) are advantageous in monitoring bimolecular interactions since they can be used to establish rate constants for both the association and dissociation phases of binding, and thus provide advantages over equilibrium-based binding measurements. Kinetic-based characterizations thus represent a preferred method for determining affinity for biological reactions such as antibody and receptor binding reactions. High affinity reactions, such as those involving high-affinity antibodies, however, pose challenges for these methods. The time period of dissociation phase of the binding can span from several hours (for nanomolar dissociation rate constants, K_{D})to several days (for picomolar dissociation constants, K_{D}). For dissociation rate measurements, a binding complex typically is formed on the sensing surface by immobilizing one of the components, and allowing the other to bind the immobilized component. For ease of discussion, we focus on antibody binding reactions, but the principles and practice of the methods described and claimed pertain to any binding reaction, including those involving other biological molecules. For antibody binding affinity measurements, an antigen typically is immobilized on the sensing surface. That surface then is exposed to a solution containing the antibody of interest, and binding proceeds. Once binding has occurred, the sensing surface is exposed to buffer solution (i.e., one that initially has no free antibody) and the dissociation rate is continuously monitored in real time. Continuously monitoring the dissociation for several hours to days as required for high-affinity antibodies occupies the instrument consequently limiting sample throughput.

Prolonged dissociation rate measurements place an additional performance demand on instrumentation to minimize baseline drift which could interfere with the rate measurement and lead to erroneous results. In solid phase instruments such as those sold by FortéBio and Biacore the solid phase presents a diffusional barrier that potentially impacts the apparent dissociation rate. As antibody dissociates from the antigen, the solid phase restricts diffusion of the antibody causing it to remain close to the antigen, thus increasing the likelihood of antibody rebinding to antigen on the surface. Rebinding of antibody on the sensing surface can lead to erroneously slow apparent dissociation rate constants. Thus, there is a need in the art for improved methods for characterizing binding reactions. The present invention provides a solution to these and other shortcomings of the prior art.

### SUMMARY OF THE INVENTION

Disclosed herein are methods and compositions for characterizing binding reactions. The method finds particular application for reactions characterized by dissociation constants in the nanomolar to picomolar range. In one aspect, the methods of the invention comprise providing in a solution a receptor and a first form of a ligand, waiting a length of time to allow the receptor and ligand to substantially reach a binding equilibrium, then adding to the solution a second form of the ligand, and determining in a solid phase binding assay a signal that arises from the binding of the first form of the ligand to the solid phase. In another aspect, the first form of the ligand specifically binds and the second form of the ligand does not specifically bind to the solid phase. In yet another aspect, substantially all of the first form of the ligand is bound to the receptor prior to the addition-of the second form of the ligand. In still another aspect, a multiple number of solid phase binding assays is carried out, in which the signals are determined at a multiple number of times following addition of the second form of the ligand. In another aspect, at least two of the times differ from each other by at least 5 hours, or by at least 10 hours, or by at least 20 hours, or by at least 100 hours. In another aspect, at least two of the determined signals differ from each other. In still another aspect, the method includes calculating a dissociation rate constant. In yet another aspect, the method includes calculating from the determined signals a ratio of bound to free first form of the ligand or an inverse of that ratio. In still another aspect, the multiple number of solid phase binding assays is carried out in the same container. In yet another aspect, the solid phase binding assay is non-destructive of the sample. In another aspect, the solid phase binding assay is a fiber-based assay. In still another aspect, the fiber-based assay comprises generating an interferometry signal. In another aspect, the solid phase binding assay is a surface plasmon resonance-based assay.

Exemplary embodiments include assays carried out using etalon-fiber based or surface plasmon resonance-based instruments.

In a variation of the invention, the reaction involves an antibody and an antigen. In another variation the two forms of the ligand differ by the inclusion of a tag that specifically binds to the solid phase. In a preferred embodiment the tag is biotin, and the solid phase includes avidin or streptavidin.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a diagram illustrating steps of the invention and results obtained carrying out the invention with an Octet™ biosensor manufactured by FortéBio, Inc.

FIG. 2 illustrates normalized data at five time points ranging from time zero to 9 hours obtained using an Octet™ biosensor with a model system in which the antibody is anti-FITC, the first form of the ligand is FITC-Dextran-Biotin, and the second form of the ligand is FITC-Dextran, fitting of those data to obtain dissociation rate constant, and comparison of the dissociation rates obtained using the methods of the invention and a prior-art assay.

FIG. 3 illustrates raw data using the same instrumentation and model system as in FIG. 2 at four different time points ranging from 10 minutes to 9 hours.

FIG. 4 illustrates raw data using the same instrumentation and model system as in FIGS. 2 and 3 obtained at sixteen different time points ranging from 29 minutes to 1763 minutes.

FIG. 5 illustrates a method of fitting the data of FIG. 4 with a single exponential.

FIG. 6 illustrates data obtained using the same instrumentation and model system as in FIGS. 2 and 3 but with two different concentrations of the second form of ligand, and linear fits of the obtained data plotted on a semi-logarithmic scale.

### DETAILED DESCRIPTION OF THE INVENTION

Advantages and Utility

Briefly, and as described in more detail below, described herein are methods for characterizing binding reactions.

Several features of the current approach should be noted. First, the methods of the invention improve the accuracy of dissociation rate constant estimation by reducing sources of error, including instrumentation drift, sample evaporation, and diffusion artifacts. The methods of the invention permit multiple measurements to be made on a single sample. In preferred embodiments using a fiber-based assay, multiple measurements can be carried out using exceedingly small volumes, thus conserving sample materials.

Advantages of this approach are numerous. They include sample conservation, improvement in accuracy of parameter estimation, and dramatic increases in measurement throughput using a single instrument.

The invention is useful for estimating the dissociation rate of a chemical reaction, and finds particular utility when the chemical reaction is characterized by tight affinity (e.g., having dissociation constants in the nanomolar to picomolar or less range), and where the dissociation rate is sufficiently slow so that dissociation occurs over intervals of hours to days or more.

Definitions

Terms used in the claims and specification are defined as set forth below unless otherwise specified.

A “solid phase binding assay” is a binding reaction in which at least one component of the reaction is affixed to a support substrate. Exemplary solid phase binding assays are assays using etalon fibers such as those carried out using the Octet™ available from FortéBio, Inc., of Menlo Park, Calif. or those carried out using a surface plasmon-resonance instrument such as those available from Biacore, Inc. of Uppsala, Sweden.

A “specific binding reaction” is one that can be shown to be saturable, and one in which binding of a labeled component can be competed with an excess of an unlabeled form of that component.

A “non-destructive assay” is an assay in which measurement from a sample can be accomplished without materially consuming the sample. Thus, a non-destructive assay is one in which repeated measurements can be obtained from a single sample.

A “fiber-based assay” is an assay in which measurements are obtained from a reaction that occurs on a fiber, such as a glass fiber.

An “interferometry signal” is a signal that arises from interference among rays of electromagnetic radiation.

An “antibody” is defined as a full length immunoglobulin molecule having two heavy chains and two light chains and which form an antigen combining site at the interface of the variable regions of the heavy and light chains. In addition, the term “antibody” is intended to encompass in addition to full length immunoglobulin molecules, fragments of the same, such as Fab fragments, as well as recombinant single chain molecules such as, e.g., scFvs.

Abbreviations used in this application include the following:

“FITC” is fluorescein isothyocyanate.

“L*” is a first form of a ligand, which specifically binds to the solid phase in a solid phase binding assay.

“L” is a second form of the ligand which does not specifically bind to the solid phase in the solid phase binding assay.

“R” is a binding partner, or receptor, which specifically binds to both L* and L.

It must be noted that, as used in the specification and the appended claims, the singular forms “a,” “an” and “the” include plural referents unless the context clearly dictates otherwise.

Methods of the Invention

The invention is directed to the problem of accurately characterizing binding reactions in which dissociation occurs very slowly, over time periods ranging from hours to days or longer. Exemplary reactions are those that occur between antibodies and antigens, especially those that are characterized by dissociation constants in the nanomolar to picomolar range, although the method can be practiced using any binding reaction in which two forms of a ligand can be prepared so that one form of the ligand specifically binds to a solid phase and a second form does not specifically bind to that solid phase. Typically the first and second forms of the ligand will differ only by the presence or absence of an affinity tag which specifically binds to the solid phase, and which does not materially interfere with the binding of the ligand to its receptor. Typical affinity tags include biotin, oligomeric histidine, oligonucleotides, lectins, etc.

For ease of illustration, the invention is described by reference to antibody binding reactions, but the scope of the invention is not limited to this example, but only by the appended claims. In a preferred embodiment, the invention can be used to solve the problem of measuring rate constants for high affinity antibodies using biosensors such as the etalon fiber-based Octet™ available from FortéBio, Inc. of Menlo Park California, or the surface plasmon-resonance-based Biacore available from Biacore, Inc. of Uppsala, Sweden. In an embodiment of the invention, a dissociation rate is determined using an assay format in which dissociation is allowed to occur in a liquid phase, and in the progress of that dissociation is monitored using a solid phase assay. The solid phase assay is carried out by contacting the solid phase with the liquid phase at one or more time intervals to obtain a signal arising from the interaction of the solid and liquid phases from which an estimate of the fraction of bound vs. free ligand can be determined.

The principle of the assay format is based on the provision of two forms of a ligand, L* and L, which differ in their ability to specifically bind to the solid phase, but which do not materially differ in their ability to bind to the binding partner present, R, in the liquid phase. In especially preferred embodiments, the concentration of the ligand form capable of specifically binding in the solid phase assay, L*, is less than the concentration of the liquid phase binding partner, R, which is in turn, less than the concentration of the ligand form not capable of specifically binding in the solid phase assay, L. The solid phase comprises a binding partner of the moiety which distinguishes L* from L. Exemplary solid phase binding partners include antibody, avidin or streptavidin, nickel, oligonucleotides (including aptamers), lectins, carbohydrate, etc., which recognize moieties such as antigen, proteins, peptides, biotin, oligomeric histidine, complementary oligonucleotides, carbohydrates, and lectins. Exemplary chemistries for derivatizing a solid for phase such as glass or plastic and for derivatizing ligands for use in the methods of the invention are well known to the ordinarily skilled practitioner and exemplary protocols are widely available in published literature and textbooks such as those listed immediately following, the entire disclosures of which are incorporated herein by reference for all purposes: Comparison of Affinity Tags for Protein Purification, 2005, 41, 98-105, Lichty et al.; Design of High-Throughput Methods of Protein Production for Structural Biology, Structure, 2000, v8, 9, R177-R185, Stevens; Protein Microarrays: Challenges and Promises, Pharmacogenomics, 2002, 3(4), 1-10, Talapatra et al.; Aptamers: Affinity Tags for the Study of RNA and Ribonuceoproteins, RNA 2001, v7(4), 632-641, Srisawat and Engelke; Immobilization of Proteins to a Carboxymethyldextran Modified Gold Surface for Bispecific Interaction Analysis in Surface Plasmon Resonance, Analytical Biochem. 1991, 198, 268-277, Johnsson et al.; Chemistry of Protein Conjugation and Crosslinking, ed. Shan Wong, by CRC Press, Boca Raton Fla., 1993; and Immunochemistry of Solid Phase Immunoassay, ed. John Butler, by CRC Press, Boca Raton, FL, 1991

In some embodiments, the binding reaction between L* and R is allowed to proceed to equilibrium before L is added to the liquid phase. In some embodiments essentially all of L* is bound to R before L is added to the liquid phase. Once L is added to the liquid phase, one or more solid phase binding assays is carried out to obtain a signal useful for characterizing the degree to which L* remains bound to R. By obtaining a number of these signals, the dissociation rate of L* from R in solution can be estimated using techniques well known to those of ordinary skill in the art.

Dissociation rate constants derived by the methods of the invention are more accurate than those obtained by direct measurement of dissociation from a solid phase. Typically, those measurements are carried out by having, e.g., R and L bound on a solid phase and exposing that solid phase to a liquid phase that has little or no free L. Such direct measurements from a solid phase are prone to error because, e.g., the solid phase can present a diffusion barrier which encourages re-binding of L to R. In contrast, the dissociation rate constants derived using the methods of the invention reflect processes occurring in liquid phase and so more closely mimic in vivo binding and avoid the diffusional barrier artifact with rate constant measurements performed with solid phase devices. The methods of the invention offer the additional advantage of not requiring continuous measurement by the biosensor instrument leaving it available to process other samples. The methods of the invention carry out measurements at only selected intervals during the dissociation phase depending on the binding affinity and time course of the dissociation phase of the reaction. Thus, the methods of the invention permit characterization of very high affinity reactions (e.g., nanomolar to picomolar or tighter equilibrium dissociation constants) having slow dissociation rates so that once L is provided, L* dissociates from R over time intervals spanning many hours to several days.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention may employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### EXAMPLE 1

### Overview of Method for Characterizing Antibody Antigen Reactions

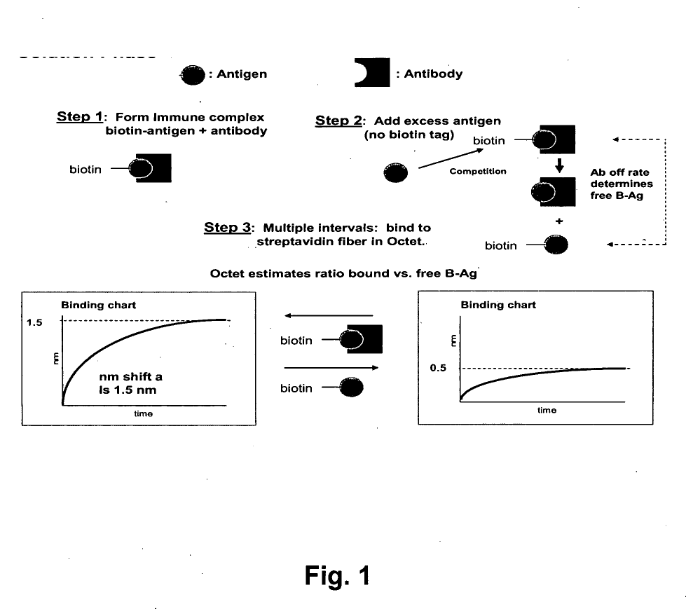

A protocol for practicing the methods of the invention to characterize an antigen-antibody reaction is illustrated in FIG. 1. In step one, a sample is prepared by forming in a standard biological buffer such as phosphate buffered saline (PBS) or any other solution appropriate binding buffer or solution, an immune complex between an affinity-tagged (e.g., biotinylated) antigen (L*) and the antibody (R) whose dissociation rate constant is to be estimated. The tagged antigen and antibody concentrations preferably are selected such that at equilibrium, essentially all of the tagged antigen is bound by the antibody. In step two, a vast molar excess of untagged antigen (L) is added to set up a competition reaction with the biotin-antigen for the antibody binding sites. In this format since the biotin-antigen has been previously bound by the antibody, the immune complex must dissociate in order for the untagged antigen to bind. Upon dissociation, the molar excess of untagged antigen favors its binding to antibody. Over time free biotin-antigen begins to appear in the sample and its rate of appearance is dependent on the antibody dissociation rate. Step 3 shows at selected time intervals immersion of a streptavidin coated biosensor in the sample mixture to measure the amount of bound versus free biotin-antigen which is subsequently used to derive a dissociation rate constant.

### EXAMPLE 2

### Characterization of Anti-FITC/FITC Reaction Using Glass Fiber Bio-layer Interferometry Sensor

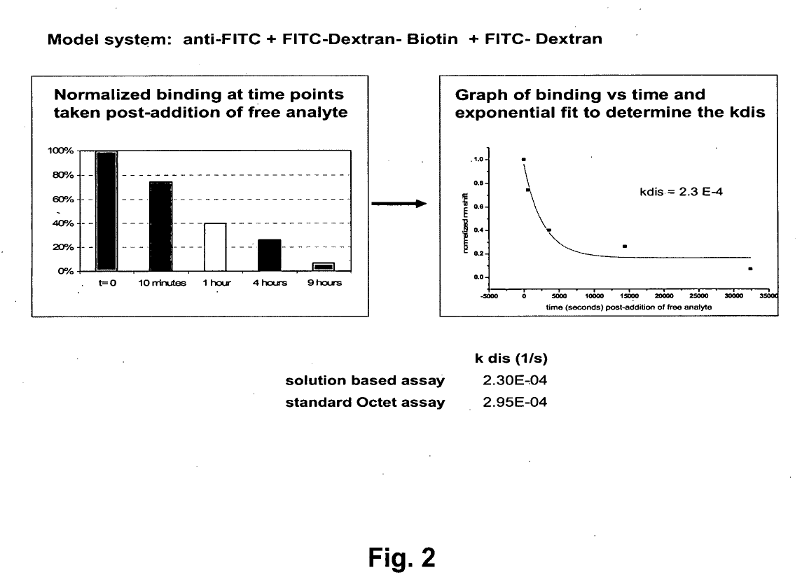

FIG. 2 depicts Step 3 dissociation results (as diagrammed in FIG. 1) for an anti fluorescein/fluorescein-dextran binding pair. In this example, a glass fiber bio-layer interferomneter sensor is used to carry out the solid phase binding assay. The sensor and methods for making and using it are described in detail in co-owned U.S. patent application publication No. 20050254062, incorporated herein by reference in its entirety for all purposes. In this example, the fiber is derivatized with streptavidin which binds to the biotin tag present on the FITC-Dextran ligand. Using this type of sensor in the practice of the method offers an unexpected advantage. At the early stages of development of therapeutic antibodies the amount of antibody can be in limited supply. Accuracy of the dissociation rate estimate is improved by making repeated measurements sensor measurements of the same sample mixture. The glass fiber bio-sensors minimize consumption of antibody samples since they are compatible with conventional sample reaction chambers, such as microtiter plate wells, requiring only 100 uL or less. Such glass fibers sensors have diameters of around 0.5 mm producing small sensing areas. Repeated measurements in the same sample are facilitated by the small sensing area with relatively limited binding capacity since the repeated measurements have a negligible impact on the total amount of biotin-antigen in the sample.

Materials & Methods

Reagents were obtained from the following sources:

Free unbiotinylated Analyte (catalog # D3306, Molecular Probes, Dextran-Fluorescein, 3000 MW)

Biotin-Analyte (catalog # D7156, Molecular Probes, Dextran-Fluorescein and Biotin, 3000 MW)

Antibody (catalog #A-6413, Molecular Probes, anti-Fluorescein antibody, Rabbit polyclonal, Fab-fragment)

K-sensors (catalog #18-0002, FortéBio)

Sample-Diluent (catalog #18-1000, FortéBio)

Experimental samples:

Biotin-Analyte (negative control; minimum signal)

Biotin-Analyte+Antibody (positive control; maximum signal)

Biotin-Analyte+Antibody+free unbiotinylated Analyte

Add different concentrations of free unbiotinylated Analyte for competition

1. Equilibrium Binding of Biotin-Analyte+Antibody:

1.1. Prepare 2× Biotin-Antigen solution in sample diluent. For this example, a solution of Biotin-Dextran-Fluorescein (0.25 μg/ml) was prepared. This stock was used for preparing the negative control as well as the Biotin+Antibody solution.

1.2. The Biotin-Antigen and Antibody (anti-Fluorescein) stocks were combined to obtain final concentrations as follows: 41.67 nM of Biotin-Antigen and 125 nM Antibody (3× molar excess of R to L*).

1.3. The negative control (Biotin-Antigen only) was prepared to obtain a concentration of 41.67 nM.

1.4. All samples were incubated overnight at room temperature with gentle agitation in order to reach equilibrium.

2. Competition assay for off-rate (dissociation rate) determination: .9

2.1. Duplicate sample sets were prepared as described below to obtain 2 assay plates for analysis using the Octet instrument available from FortéBio, Inc.

2.2. 100× and 1000× molar excess (above Antibody, R) of free unbiotinylated Antigen (Dextran-Fluorescein, L) was added to 2 aliquots of the solution from 1.2. The final concentrations of free unbiotinylated Antigen (L) in the two samples were respectively 12.5 μM and 125 μM.

2.3. The total volume added to each reaction was kept equivalent in all samples by adjusting the total volume with PBS.

2.4. PBS was added to the negative control instead of free unbiotinylated Antigen (L) to obtain the same total volume as samples in 2.1.

2.5. The assay plate: Black Flat bottom 96-well PP plate from Greiner (E&K Scientific # EK-25209)

2.5.1. Wells in column 1 were filled with sample diluent for baseline determination (200 μl each).

2.5.2. Wells in column 2 were filled with sample solutions (200 μl each).

3. Assay on the FortéBio Octet:

3.1. The first time points were taken directly after the incubations were started.

3.2. Assay protocol using the Octet and K sensors:

3.2.1. Sample plates were warmed to 30° C. in the Octet for 5 minutes (no cover on plate).

3.2.2. Assay protocol was set up as follows:

3.2.2.1. Baseline in column 1 sample diluent was obtained for 60 seconds with plate agitating at 200 rpm and temperature held at 30° C.

3.2.2.2. Binding data was obtained from wells in column 2 for 1200 seconds with plate agitating at 200 rpm and temperature held at 30° C.

3.2.3. After the initial binding data were obtained, the sample plates were removed from the Octet, sealed with a plate sealer and incubated on the laboratory bench at ambient temperature. One plate was used for all subsequent time points.

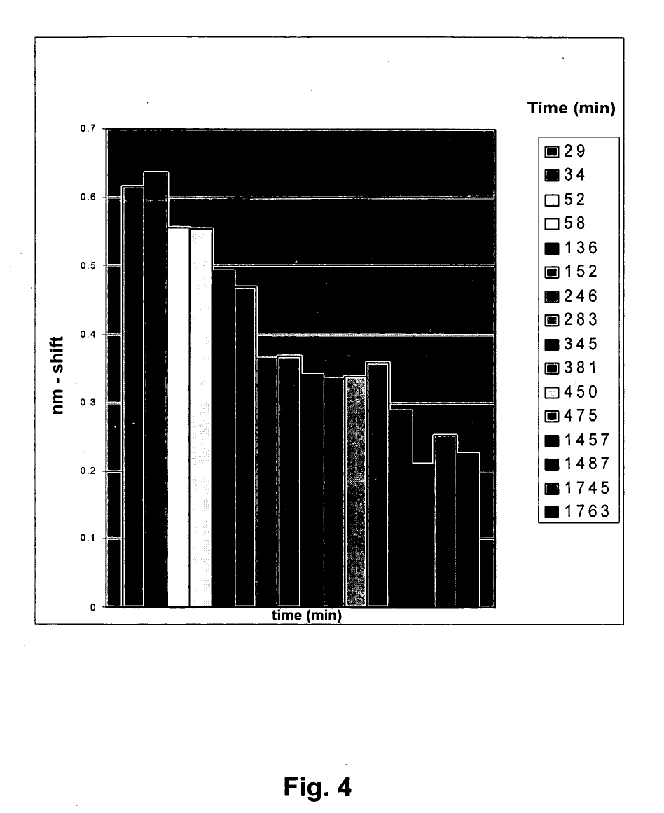

3.3. Steps in 3.2 were repeated at each time point. For the example data shown, data was taken at 14, 38, 132, 226, 361, 455, 1467, 1783 minutes for plate 1 and 9, 32, 116, 263, 325, 430, 1437, 1725 minutes for duplicate plate 2. Exemplary data are shown in FIG. 4 which provides data from both experiments Note that the times provided in the legend to FIG. 4 are offset by 20 minutes from the values provided in the paragraph which represents the amount of time that binding to the solid phase was allowed to proceed.

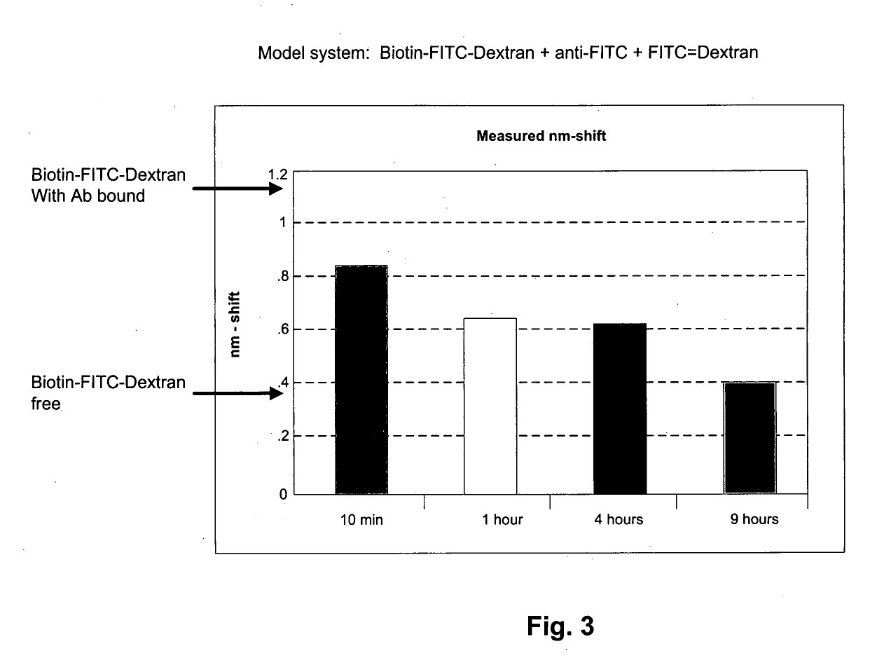

FIG. 2 (left side) shows representative normalized data obtained using the above-described protocol, and its analysis according to the first analytic method described below to estimate the dissociation rate constant (right side). FIG. 3 shows representative data obtained at time points following addition of L, as well as maximum binding of RL* (positive control) and minimum binding of L* (negative control)

4. Data analysis:

4.1. The total nm-shift for each measurement was determined at the end of the assay. In this example the nm-shift was determined for each time point following a 20 minute binding period to the sensor tip. That 20 minute period was chosen because it provided a robust readout and was empirically determined to be a time point that effectively reported the endpoint of binding of the solution phase components to the solid phase biosensor. Of course, in other applications, the binding period time may differ as a function of parameters such as, e.g., temperature, ionic, strength, and the diffusion constants for the RL* and L* components, and suitable binding period time can readily be determined by the ordinarily skilled artisan having the benefit of this disclosure.

Although in this example the binding data for each time point was reported in the form of a nm shift that developed over a 20 minute period, the methods also can be practiced by using the initial shift that develops over, e.g., a shorter period, such as, one to several minutes, or the first derivative (e.g., rate) of binding taken over an initial binding period. An advantage of using the initial shift or initial rate is that it minimizes the likelihood that significant further dissociation of the RL* complex occurs during the course of the analysis.

4.2. Dissociation rate constant, k_{dis} determination method 1:

4.2.1. For each sample, a graph was created of time versus nm shift (X vs.Y). The average of the positive Biotin-Antigen+Antibody control was used for the data point for nm-shift at time 0.

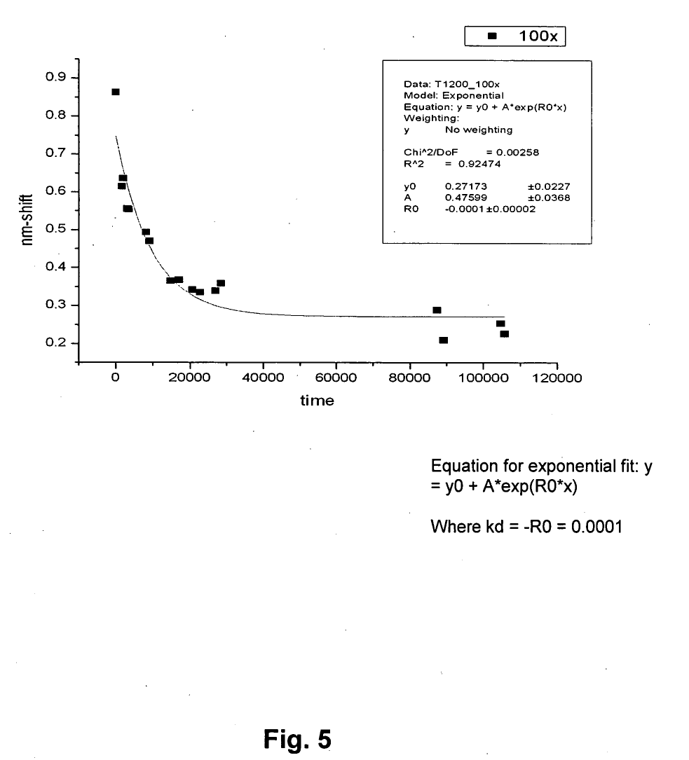

4.2.2. An exponential fit of these data were used to determine k_{dis}, where y=y0+A*exp(R0*x). k_{dis}=−R0. Examples of these determinations are provided in the right hand side of FIG. 2 and in FIG. 5.

4.3. k_{dis} determination method 2:

4.3.1. For each sample, a graph was created of the ln(time) vs nm shift (X vs Y) and the semi-logarithmically plotted data were linearly fitted.

4.3.2. From the linear fits, we determined the time at which half the signal (nm shift) was lost. This is the t½.

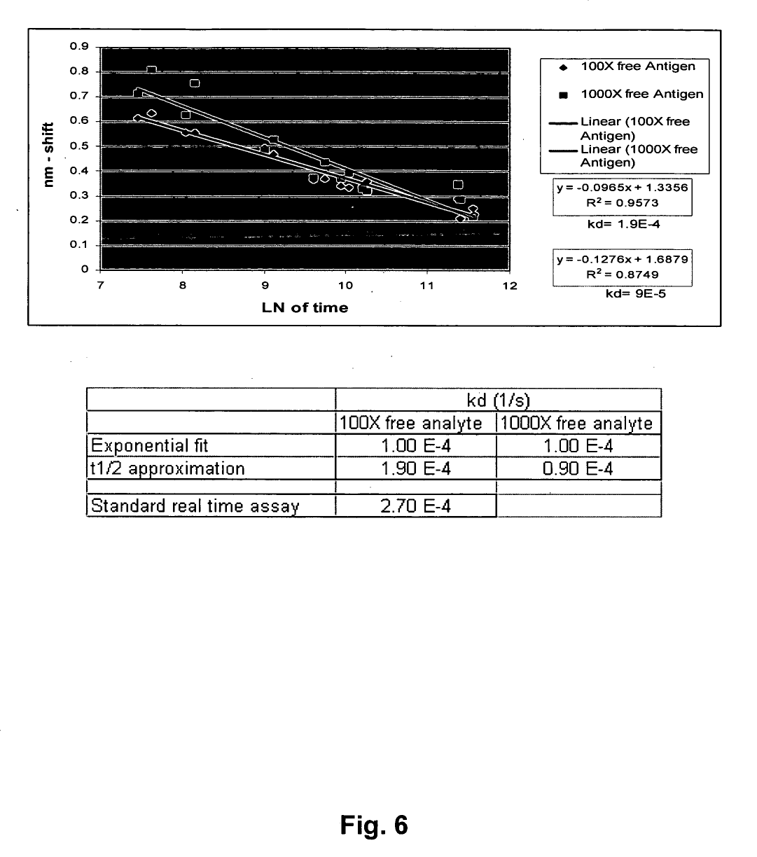

4.3.3. The k_{dis} was approximated by the equation k_{dis}=(ln2/t½), and is shown in the top part of FIG. 6 for the 100-fold and 1000-fold molar excess competitions. The bottom part of FIG. 6 tabulates the estimated dissociation rate constants (denoted here as kd instead of k_{dis}) using both estimation methods as well as the rate obtained using a standard Octet real time assay.

### EXAMPLE 3

### Implementation of the Methods of the Invention on a BIAcore Device

1. Sample setup is carried out in a manner similar to that described in Example 2, except that samples are prepared in a large batch (e.g., 10-20 mL). Each assay consumes one aliquot (e.g., on the order of 1-2 mL). In addition a solution control is used to correct for any refractive index changes due to protein concentration. For purposes of this working example, the ideal solution control consists of the unbiotinylated analyte plus antibody to give a similar total protein concentration as the samples.

2. Streptavidin Biacore chips are used (Sensor chip SA, Biacore cat number BR-1000-32). As an alternative, an amine reactive CM5 chip (Biacore cat number BR-1006-68) can be used to immobilized streptavidin through a standard protocol.

3. For each time point, a new Biacore chip is used. Each chip contains four flowcells that are used to assay the three samples described in Example 2 plus the solution control.

4. At each time point one aliquot of each solution is injected over a separate flowcell.

5. At the next time point a new chip is inserted into the instrument and a new aliquot of each of the samples and solution control is injected.

9. Once all time points are taken, data analysis proceeds as described in Example 2, except the Biacore RU values (Biacore signal units) are used in place of the Octet nm shift.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method for characterizing a reaction, comprising:
providing a substantially equilibrated solution comprising a receptor at a total concentration [R] and a first form of a ligand at a total concentration [L*];
adding to said solution a second form of said ligand at a total concentration [L]; and
determining in a solid phase binding assay a signal arising from the binding of said first form of said ligand to said solid phase at a first time after adding said second form of said ligand, wherein said first form of said ligand specifically binds, and said second form of said ligand does not specifically bind to said solid phase.

2. The method of claim 1, wherein [L*]<[R]<[L].

3. The method of claim 1, wherein prior to said addition of said second form of said ligand, substantially all of said first form of said ligand is bound to said receptor.

4. The method of claim 1, further comprising determining in a plurality of solid phase binding assays a plurality of signals arising from the binding of said first form of said ligand to said solid phase, wherein said signals are determined at a plurality of times after adding said second form of said ligand.

5. The method of claim 4, wherein at least two of said signals differ from each other.

6. The method of claim 4, wherein at least two of said plurality of times differ from each other by at least 5 hours.

7. The method of claim 6, wherein at least two of said plurality of times differ from each other by at least 10 hours.

8. The method of claim 7, wherein at least two of said plurality of times differ from each other by at least 20 hours.

9. The method of claim 8, wherein at least two of said plurality of times differ from each other by at least 100 hours.

10. The method of claim 4, wherein said plurality of solid phase binding assays is carried out in a single container containing said solution.

11. The method of claim 4, further comprising calculating a dissociation rate constant from said plurality of signals determined in said plurality of solid phase binding assays.

12. The method of claim 4, further comprising calculating from said plurality of signals determined in said plurality of solid phase binding assays, a ratio of bound to free L* or an inverse of said ratio.

13. The method of claim 1, wherein said solid phase binding assay is non-destructive.

14. The method of claim 13, wherein said solid phase binding assay is a fiber-based assay.

15. The method of claim 14, wherein said fiber-based assay comprises generating an interferometry signal.

16. The method of claim 13, wherein said solid phase binding assay is a surface plasmon resonance-based assay.
